(19) United States
(12) Patent Application Publication
(10) Pub. No.: US 2010/0249589 A1
(43) Pub. Date: Sep. 30, 2010

(54) SYSTEM AND METHOD FOR FUNCTIONAL ULTRASOUND IMAGING

(76) Inventors: Peter Lysyansky, Haifa (IL); Zvi Friedman, Kiriat Bialik (IL); Andreas Heimdal, Oslo (NO); Gunnar Hansen, Vestfold (NO)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 12/410,924

(22) Filed: Mar. 25, 2009

(51) Int. Cl. A61B 8/14

(52) U.S. Cl. 600/440

(57) ABSTRACT

A system and method for functional ultrasound imaging are provided. The method includes obtaining ultrasound image data acquired from a multi-plane imaging scan of an imaged object. The ultrasound image data defines a plurality of image planes. The method also includes determining functional image information for the imaged object from two-dimensional tracking information based on the plurality of image planes and generating functional ultrasound image data for the imaged object using the functional image information.

### BACKGROUND OF THE INVENTION

This invention relates generally to diagnostic imaging systems, and more particularly, to ultrasound imaging systems providing anatomical functional imaging, especially for cardiac imaging.

Medical imaging systems are used in different applications to image different regions or areas (e.g., different organs) of patients. For example, ultrasound systems are finding use in an increasing number of applications, such as to generate images of the heart. These images are then displayed for review and analysis by a user. When imaging a heart, a sonographer typically acquires several different images of the heart along three different imaging planes. For example, when imaging the left ventricle, these include three standard images that are acquired from three different imaging planes. The three images may be combined to generate a combined image that shows the function of the entire myocardium or left ventricle. The process of acquiring the multiple images can be time consuming and may require a skilled sonographer to identify specific points (e.g., apical points) in each of the images to properly align the images when the images are combined. Moreover, the sonographer has to name each of the images to avoid confusion. If the specific points or landmarks in the images are not properly identified, the combined image of the function of the myocardium may not be entirely accurate.

Systems are also known that perform imaging to generate functional information, for example of the myocardium, using three-dimensional tracking. The processing of three-dimensional image data to generate images showing function information is more computational intensive and accordingly more time consuming. The images that results from the three-dimensional tracking also may be less robust and more difficult to interpret.

### BRIEF DESCRIPTION OF THE INVENTION

In accordance with an embodiment of the invention, a method for functional ultrasound imaging is provided. The method includes obtaining ultrasound image data acquired from a multi-plane imaging scan of an imaged object. The ultrasound image data defines a plurality of image planes. The method also includes determining functional image information for the imaged object from two-dimensional tracking information based on the plurality of image planes and generating functional ultrasound image data for the imaged object using the functional image information.

In accordance with another embodiment of the invention, a computer readable medium is provided having computer readable code readable by a machine and with instructions executable by the machine to perform a method of functional imaging. The method includes accessing multi-plane ultrasound image data of an imaged object and performing two-dimensional tracking using the multi-plane ultrasound image data. The method also includes determining functional image information based on the two-dimensional tracking and generating functional ultrasound image data using the functional image information.

In accordance with yet another embodiment of the invention, an ultrasound imaging system is provided that includes an ultrasound probe configured to perform multi-plane ultrasound imaging to acquire a plurality of image frames. The ultrasound imaging system also includes a processor having a functional imaging module configured to determine functional image information from two-dimensional tracking information for the acquired plurality of image frames and generate functional ultrasound image data.

### BRIEF DESCRIPTION OF THE DRAWINGS

FIG. 1 is a block diagram of a diagnostic ultrasound system configured to perform functional imaging in accordance with various embodiments of the invention.

FIG. 2 is a block diagram of an ultrasound processor module of the diagnostic ultrasound system of FIG. 1 formed in accordance with various embodiments of the invention.

FIG. 3 is a flowchart of method for performing functional imaging using multi-plane image acquisition in accordance with various embodiments of the invention.

FIG. 4 is a diagram illustrating image data that may be obtained from a tri-plane image scan using three planes in accordance with various embodiments of the invention.

FIG. 5 is a diagram illustrating image data that may be obtained from an image scan using six planes in accordance with various embodiments of the invention.

FIG. 6 is a display formatted as a bullseye plot showing functional information generated in accordance with various embodiments of the invention.

FIG. 7 is a diagram illustrating workflow for the functional imaging of a heart using multi-plane data acquisition with two-dimensional (2D) tracking in accordance with various embodiments of the invention.

FIG. 8 illustrates a three-dimensional capable miniaturized ultrasound system formed in accordance with an embodiment of the invention.

FIG. 9 illustrates a hand carried or pocket-sized ultrasound imaging system formed in accordance with an embodiment of the invention.

FIG. 10 illustrates a console type ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Exemplary embodiments of ultrasound systems and methods for functional imaging are described in detail below. In particular, a detailed description of an exemplary ultrasound system will first be provided followed by a detailed description of various embodiments of methods and systems for functional ultrasound imaging, especially cardiac functional ultrasound imaging.

At least one technical effect of the various embodiments of the systems and methods described herein include generating functional ultrasound images of a heart using a three-dimensional (3D) scan mode or 3D ultrasound probe. The various embodiments provide functional imaging using two-dimensional (2D) tracking applied to multiple image planes acquired simultaneously consecutively or within a short period of time using a 3D probe. The functional imaging provides an improved and more effective workflow that is less computationally intensive. Using the various embodiments, lateral imaging resolution may be increased, resulting in increased diagnostic accuracy.

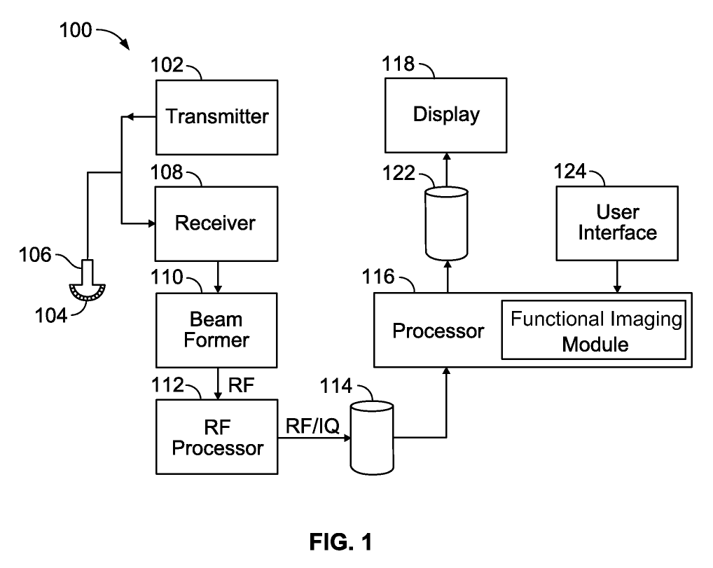

FIG. 1 is a block diagram of an ultrasound system 100 constructed in accordance with various embodiments of the invention. The ultrasound system 100 is capable of steering a soundbeam in 3D space, and is configurable to acquire information corresponding to a plurality of 2D representations or images of a region of interest (ROI) in a subject or patient. One such ROI may be the human heart or the myocardium of a human heart. The ultrasound system 100 is configurable to acquire 2D images in three or more planes of orientation.

The ultrasound system 100 includes a transmitter 102 that, under the guidance of a beamformer 110, drives an array of elements 104 (e.g., piezoelectric elements) within a probe 106 to emit pulsed ultrasonic signals into a body. A variety of geometries may be used. The ultrasonic signals are back-scattered from structures in the body, like blood cells or muscular tissue, to produce echoes that return to the elements 104. The echoes are received by a receiver 108. The received echoes are passed through the beamformer 110, which performs receive beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to a memory 114 for storage.

In the above-described embodiment, the beamformer 110 operates as a transmit and receive beamformer. In an alternative embodiment, the probe 106 includes a 2D array with sub-aperture receive beamforming inside the probe. The beamformer 110 may delay, apodize and sum each electrical signal with other electrical signals received from the probe 106. The summed signals represent echoes from the ultrasound beams or lines. The summed signals are output from the beamformer 110 to an RF processor 112. The RF processor 112 may generate different data types, e.g. B-mode, color Doppler (velocity/power/variance), tissue Doppler (velocity), and Doppler energy, for multiple scan planes or different scanning patterns. For example, the RF processor 112 may generate tissue Doppler data for three (tri-plane) scan planes. The RF processor 112 gathers the information (e.g. I/Q, B-mode, color Doppler, tissue Doppler, and Doppler energy information) related to multiple data slices and stores the data information with time stamp and orientation/rotation information in an image buffer 114.

Orientation/rotation information may indicate the angular rotation of one data slice with respect to a reference plane or another data slice. For example, in a tri-plane implementation wherein ultrasound information is acquired substantially simultaneously or consecutively within a short period of time (e.g. 1/20 second) for three differently oriented scan planes or views, one data slice may be associated with an angle of 0 degrees, another with an angle of 60 degrees, and a third with an angle of 120 degrees. Thus, data slices may be added to the image buffer 114 in a repeating order of 0 degrees, 60 degrees, 120 degrees, . . . , 0 degrees, 60 degrees, and 120 degrees, . . . . The first and fourth data slices in the image buffer 114 have a first common planar orientation. The second and fifth data slices have a second common planar orientation and third and sixth data slices have a third common planar orientation. More than three data slices may be acquired as described in more detail herein.

Alternatively, instead of storing orientation/rotation information, a data slice sequence number may be stored with the data slice in the image buffer 114. Thus, data slices may be ordered in the image buffer 114 by repeating sequence numbers, e.g. 1, 2, 3, . . . , 1, 2, 3, . . . . In tri-plane imaging, sequence number 1 may correspond to a plane with an angular rotation of 0 degrees with respect to a reference plane, sequence number 2 may correspond to a plane with an angular rotation of 60 degrees with respect to the reference plane, and sequence number 3 may correspond to a plane with an angular rotation of 120 degrees with respect to the reference plane. The data slices stored in the image buffer 114 are processed by 2D display processors as described in more detail herein.

In operation, real-time ultrasound multi-plane imaging using a matrix or 3D ultrasound probe may be provided. For example, real-time ultrasound multi-plane imaging may be performed as described co-pending U.S. patent application Ser. No. 10/925,456 entitled “METHOD AND APPARATUS FOR REAL TIME ULTRASOUND MULTI-PLANE IMAGING” commonly owned, the entire disclosure of which is hereby incorporated by reference in its entirety.

The ultrasound system 100 also includes a processor 116 to process the acquired ultrasound information (e.g., RF signal data or IQ data pairs) and prepare frames of ultrasound information for display on display 118. The processor 116 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in memory 114 during a scanning session and then processed and displayed in an off-line operation.

The processor 116 is connected to a user interface 124 that may control operation of the processor 116 as explained below in more detail. The processor 116 also includes a functional imaging module 126 that performs 2D tracking using the multi-plane imaging as described in more detail herein.

The display 118 includes one or more monitors that present patient information, including diagnostic ultrasound images to the user for diagnosis and analysis (e.g., functional images of the heart, such as a bullseye image). One or both of memory 114 and memory 122 may store three-dimensional data sets of the ultrasound data, where such 3D data sets are accessed to present 2D (and/or 3D images) as described herein. The images may be modified and the display settings of the display 118 also manually adjusted using the user interface 124.

It should be noted that although the various embodiments may be described in connection with an ultrasound system, the methods and systems described herein are not limited to ultrasound imaging or a particular configuration thereof. In particular, the various embodiments may be implemented in connection with different types of imaging, including, for example, magnetic resonance imaging (MRI) and computed-tomography (CT) imaging or combined imaging systems. Further, the various embodiments may be implemented in other non-medical imaging systems, for example, non-destructive testing systems.

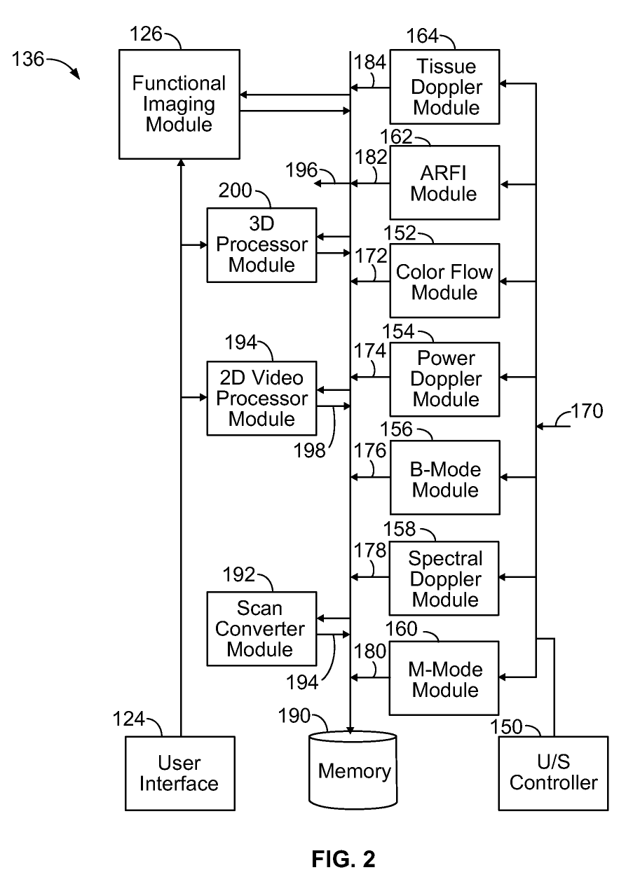

FIG. 2 illustrates an exemplary block diagram of an ultrasound processor module 136, which may be embodied as the processor 116 of FIG. 1 or a portion thereof. The ultrasound processor module 136 is illustrated conceptually as a collection of sub-modules, but may be implemented utilizing any combination of dedicated hardware boards, DSPs, processors, etc. Alternatively, the sub-modules of FIG. 2 may be implemented utilizing an off-the-shelf PC with a single processor or multiple processors, with the functional operations distributed between the processors. As a further option, the sub-modules of FIG. 2 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 2 may be controlled by a local ultrasound controller 150 or by the processor module 136. The sub-modules 152-164 perform mid-processor operations. The ultrasound processor module 136 may receive ultrasound data 170 in one of several forms. In the embodiment of FIG. 2, the received ultrasound data 170 constitutes I,Q data pairs representing the real and imaginary components associated with each data sample. The I,Q data pairs are provided to one or more of a color-flow sub-module 152, a power Doppler sub-module 154, a B-mode sub-module 156, a spectral Doppler sub-module 158 and an M-mode sub-module 160. Optionally, other sub-modules may be included such as an Acoustic Radiation Force Impulse (ARFI) sub-module 162 and a Tissue Doppler (TDE) sub-module 164, among others.

Each of sub-modules 152-164 are configured to process the I,Q data pairs in a corresponding manner to generate color-flow data 172, power Doppler data 174, B-mode data 176, spectral Doppler data 178, M-mode data 180, ARFI data 182, and tissue Doppler data 184, all of which may be stored in a memory 190 (or memory 114 or memory 122 shown in FIG. 1) temporarily before subsequent processing. For example, the B-mode sub-module 156 may generate B-mode data 176 including a plurality of B-mode image planes, such as in a triplane image acquisition as described in more detail herein.

The data 172-184 may be stored, for example, as sets of vector data values, where each set defines an individual ultrasound image frame. The vector data values are generally organized based on the polar coordinate system.

A scan converter sub-module 192 access and obtains from the memory 190 the vector data values associated with an image frame and converts the set of vector data values to Cartesian coordinates to generate an ultrasound image frame 194 formatted for display. The ultrasound image frames 194 generated by the scan converter module 192 may be provided back to the memory 190 for subsequent processing or may be provided to the memory 114 or the memory 122.

Once the scan converter sub-module 192 generates the ultrasound image frames 194 associated with, for example, B-mode image data, and the like, the image frames may be restored in the memory 190 or communicated over a bus 196 to a database (not shown), the memory 114, the memory 122 and/or to other processors, for example, the functional imaging module 126.

As an example, it may be desired to view functional ultrasound images or associated data (e.g., strain curves or traces) relating to echocardiographic functions on the display 118 (shown in FIG. 1). Strain information for display as part of the functional ultrasound images are calculated based on scan converted B-mode images. The scan converted data is then converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 118 (shown in FIG. 1), which may include one or more monitors or windows of the display, to display the image frame. The echocardiographic image displayed in the display 118 is produced from image frames of data in which each datum indicates the intensity or brightness of a respective pixel in the display. In this example, the display image represents muscle motion in a region of interest being imaged based on 2D tracking applied to a multi-plane image acquisition as described in more detail herein.

Referring again to FIG. 2, a 2D video processor sub-module 194 combines one or more of the frames generated from the different types of ultrasound information. For example, the 2D video processor sub-module 194 may combine a different image frames by mapping one type of data to a grey map and mapping the other type of data to a color map for video display. In the final displayed image, color pixel data may be superimposed on the grey scale pixel data to form a single multi-mode image frame 198 (e.g., functional image) that is again re-stored in the memory 190 or communicated over the bus 196. Successive frames of images may be stored as a cine loop in the memory 190 or memory 122 (shown in FIG. 1). The cine loop represents a first in, first out circular image buffer to capture image data that is displayed to the user. The user may freeze the cine loop by entering a freeze command at the user interface 124. The user interface 124 may include, for example, a keyboard and mouse and all other input controls associated with inputting information into the ultrasound system 100 (shown in FIG. 1).

A 3D processor sub-module 200 is also controlled by the user interface 124 and accesses the memory 190 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

The functional imaging module 126 is also controlled by the user interface 124 and accesses the memory 190 to obtain ultrasound information, and as described in more detail below, use multiple image planes, for example, acquired by a 3D probe to generate functional images of the heart using 2D tracking.

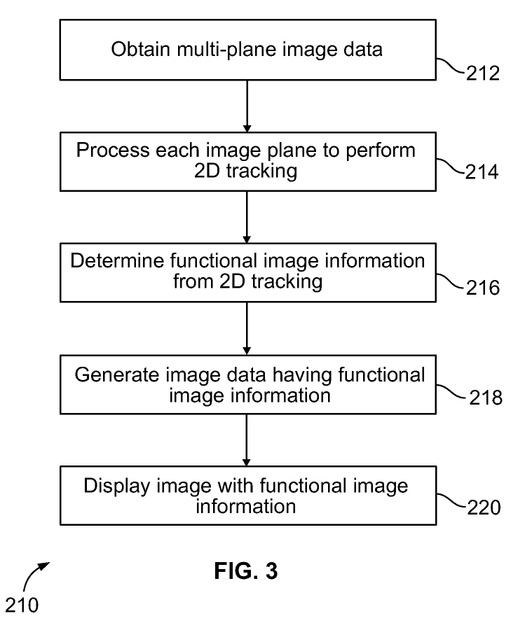

More particularly, a method 210 for performing functional imaging using multi-plane image acquisition is shown in FIG. 3. It should be noted that although the method 210 is described in connection with ultrasound imaging having particular characteristics, the various embodiments are not limited to ultrasound imaging or to and particular imaging characteristics.

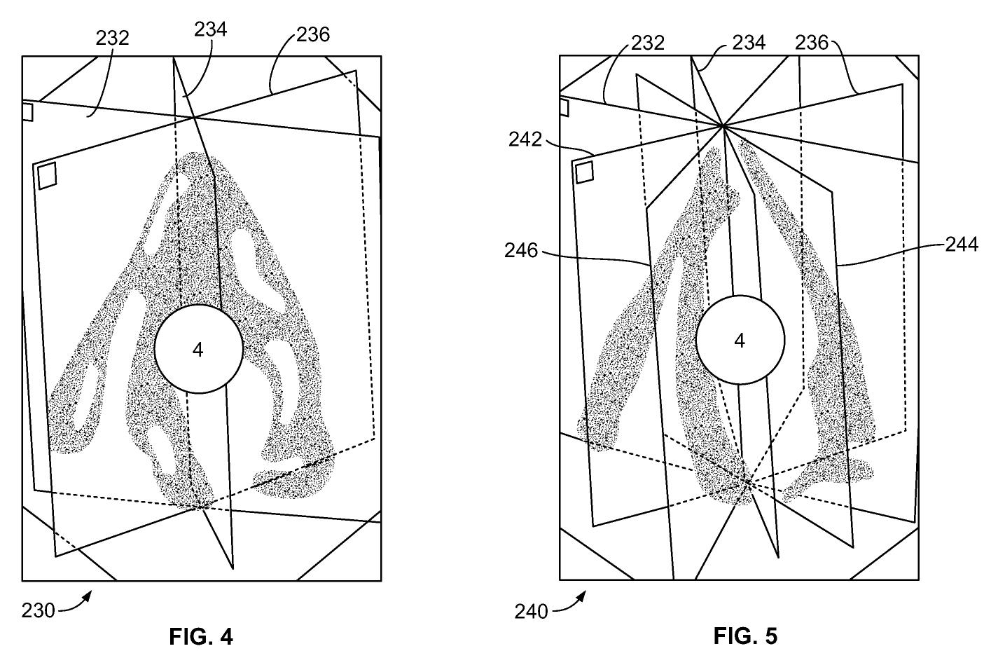

The method 210 includes obtaining multi-plane image data at 212. The multi-plane image data may be obtained from a current image scan or from previously obtained and stored data. In some embodiments, the multi-plane image data is acquired from a 3D ultrasound scan using two or more image planes. For example, as shown in FIG. 4, the image data 230 may be obtained from a tri-plane image scan using three-planes (tri-plane imaging) 232, 234 and 236. It should be noted that each of the scan planes is a 2D scan plane. Additionally, it should be noted that the multi-plane image acquisition may be performed using any type of ultrasound probe and/or ultrasound imaging system as appropriate. For example, the multi-plane imaging may be performed using the Vivid line of ultrasound systems, such as the Vivid 7 or Vivid E9 available from GE Healthcare.

In some embodiments, such as in a tri-plane image acquisition implementation, ultrasound information is acquired substantially simultaneously or consecutively within a short period of time (e.g. 1/20 second) for the three differently oriented scan planes 232, 234 and 236 or views. It should be noted that the spacing (e.g., angular rotation) between the scan planes 232, 234 and 236 may be the same or varied. For example, one data slice associated with scan plane 232 may correspond to an angle of 0 degrees, another data slice associated with scan plane 234 may correspond to an angle of 60 degrees, and a third data slice associated with scan plane 236 may correspond to an angle of 120 degrees.

A 2D combined image, a 3D combined image or other image may be formed from the image planes (e.g., individual planes of a multi-plane dataset). The scan planes 232, 234 and 236 may intersect at a common rotational axis 238 or, alternatively, intersect at different axes. Three slice images (e.g., 2D slices cut through a full volume 3D dataset) may be generated by image data acquired at the three scan planes 232, 234 and 236, which are three views of the scan object at about the same point in time due to simultaneous acquisition of the scan data for the three scan planes 232, 234 and 236. The three slice images may be, for example, of a patient's heart at a specific point in time of the heart beat or cycle. Alternatively, the three slice images may show continuous motion of a patient's heart while the heart beats. It should be noted that one or more of the scan planes 232, 234 and 236 may be tilted relative to a scanning surface of the ultrasound probe 106 (shown in FIG. 1). Additionally, the angular rotation between the scan planes 232, 234 and 236 may be changed or varied.

It also should be noted that the scan planes 232, 234 and 236 may be acquired by mechanical or electronic steering of an ultrasound probe. For example, in some embodiments, the ultrasound probe may include a mechanically movable scan head as is known that moves the array of elements 104 (shown in FIG. 1) to acquire image data (e.g., image planes) corresponding to the scan planes 232, 234 and 236. In other embodiments, the ultrasound probe may include electronic steering means as is known that electronically steers a matrix array to acquire the image data corresponding to the scan planes 232, 234 and 236. In still other embodiments, a combination of mechanical and electronic steering as is known may be used. It should be noted that during acquisition of the scan planes 232, 234 and 236, the probe housing in various embodiments is not moved relative to the object being examined.

It also should be noted that more than three scan planes may be used to acquire image information. For example, six images (e.g., six image planes) may be generated by image data 240 acquired at the six planes, namely, scan planes 232, 234 and 236, as well as scan planes 242, 244 and 246, which may located, for example, equidistance between the scan planes 232, 234 and 236 as shown in FIG. 5. Accordingly, each of the scan planes 232, 234, 236, 242, 244 and 246 may each be separated by thirty degrees. However, the angular spacing between each of the scan planes may be varied. Accordingly, the number of apical planes may be increased using, for example, sequentially acquired multi-plane scan data by electronically rotating the scan angles. In some embodiments, multiple tri-plane acquisitions may be performed that are angularly rotated with respect to each other or a single acquisition having more than three scan planes may be performed. Thus, increased image resolution of, for example, the left ventricle of an imaged heart may be provided.

Referring again to the method 210 shown in FIG. 3, after the multi-plane image data is obtained at 212, each image plane is processed at 214 to perform 2D tracking. For example, in some embodiments, each image plane is processed such that quantitative analysis of left ventricle function is performed, such as by performing 2D speckle tracking. It should be noted that the tracking may be performed from acquired apical views. Additionally, it should be noted that a normal left ventricle will display the lowest motion at the apex, while the mitral annulus will display the greatest motion. Also it should be noted that systolic mitral annular displacement, determined by the tracking, correlates closely with left ventricular ejection fraction.

The various processing function performed on each plane generally tracks in 2D, based on image data from the scan planes, the motion of the heart, and in particular, the myocardium or left ventricle, such as longitudinal displacement The processing functions may be performed using, for example, the Vivid line of ultrasound systems available from GE Healthcare. In general, the processing of each image plane, which may define different image frames, may be performed using any known method that determines or tracks motion of the heart, particularly of the myocardium or left ventricle.

After each image plane has been processed at 214, functional image information from the 2D tracking is determined at 216. For example, ventricular wall motion may be determined from the 2D tracking. The wall motion information may be quantified based on the measured movement of the ventricular wall. For example, an automated function imaging process may be performed using the Vivid™ 7 Dimension system and/or EchoPAC™ workstation available from GE Healthcare. The automated function imaging facilitates assessing left ventricular function at rest to perform quantitative assessment to determine potential wall motion abnormalities.

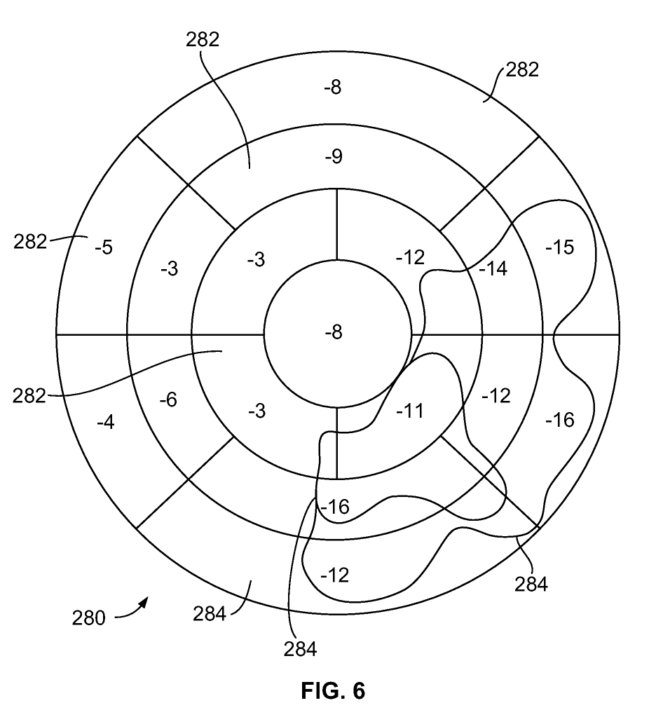

Using the determined functional information, image data including the functional image information is generated at 218 and may optionally be displayed at 220. For example, after generating the image data including the functional information, a display 280 as shown in FIG. 6 may be generated and displayed. The display 280 is configured as a bullseye plot having a plurality of segments 282 as is known (17 segments are shown, but more or less segments, for example, 16 segments or 18 segments may be provided). Each of the segments 282 may include therein a numeric value indicating the peak systolic strain for that segment 282. Additionally, color coded regions 284 may be provided that indicate the amount of contraction. For example, the regions 284 may generally indicate an estimated spatial and temporal behavior of the left ventricle by showing a distribution of the contraction of the myocardium. Different colors may represent different levels of heart wall motion or contraction.

However, the various embodiments are not limited to a particular type of display. For example, strain traces or images, or curved anatomical M-mode images may be displayed showing the functional information as is known (e.g., color coded functional information).

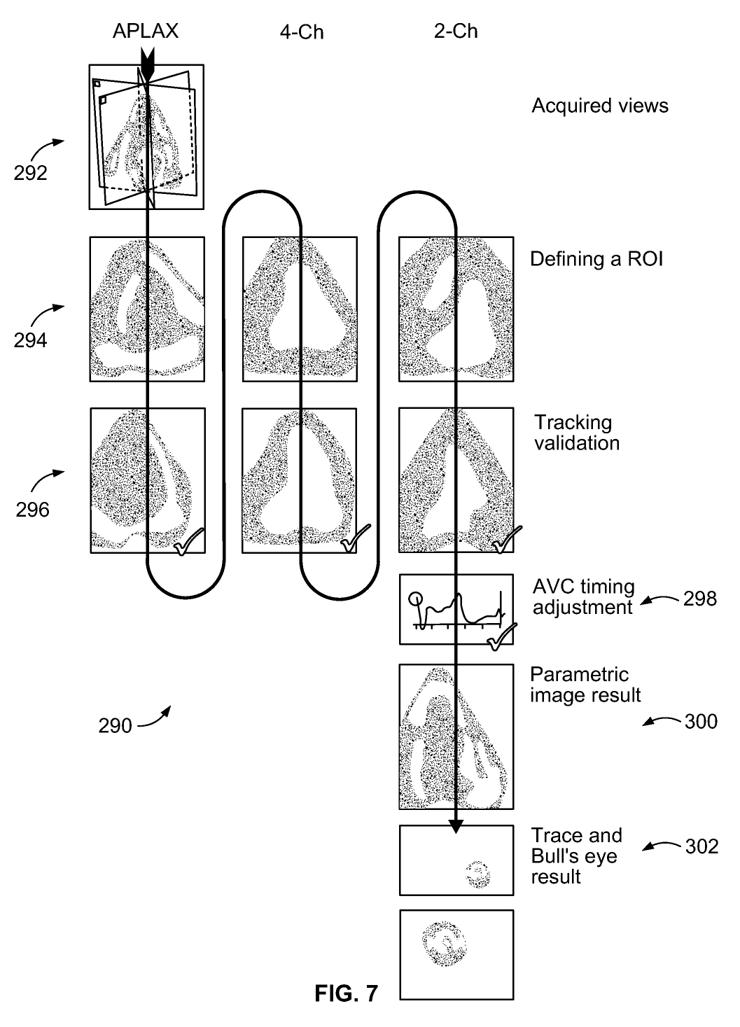

Various embodiments of the invention provide functional imaging, for example, automated functional imaging using 2D tracking based on multi-plane data acquisition using, for example, a 3D ultrasound scan. The various embodiments provide, for example, automated functional imaging as shown in FIG. 7, which illustrates a workflow 290 for the functional imaging of a heart using multi-plane data acquisition with 2D tracking. It should be noted that the workflow 290 may be performed in hardware, software or a combination thereof.

The workflow includes acquiring multiple views or data slices using a multi-plane ultrasound scan at 292. It should be noted that the number of planes used to acquire the ultrasound data may be any number, for example, two or more as described herein. As described herein, three scan planes may be automatically acquired, for example, using electronic beam steering. The three scan planes may be, for example, standard views such as an apical long axis view, a 4-chamber view and a 2-chamber view of the heart. A region of interest, for example, the left ventricle or myocardium is defined at 294. It should be noted that the region of interest is identified for each scan plane. The region of interest may be defined by identifying one or more landmarks, for example, the apical point of the myocardium, which may be manually identified by a user (e.g., by pointing and clicking with a mouse) or automatically identified, such as by using know movements within the heart. However, it should be noted that because the left ventricle long axis orientation is defined by the multi-plane scan, the apical point position for all scan planes can be automatically determined (e.g., based on the known angular rotation of each of the scan planes). For example, once a single apical point is determined on a single view, for example, by a user or automatically, the apical point is defined for all scan planes.

In some embodiments, automatic apical point detection may be provided in any suitable manner. For example, a user may identify one or more anatomical landmarks (e.g., mitral valve annulus), which is then used to automatically identify the apical point, such as based on a known distance from the anatomical landmark. As another example, motion within the image may be used to automatically determine the apical point, such as based on a known distance from an identified moving portion of the heart.

After the region of interest in defined, tracking validation is performed at 296, which is performed for each image frame. For example, the image quality or 2D tracking quality as described in more detail herein may be validated by a user or compared to a model image to determine if the image is within a predetermined variance. If the quality is not acceptable, the image data may be reprocessed. Additionally, it should be noted that segments of the myocardium that do not satisfy a certain quality level may be excluded from the displayed results (e.g., gray color coding on the bullseye plot). Thereafter, aortic valve closure (AVC) adjustment may be performed at 298. For example, a user may confirm the AVC on the long axis apical view as is known to ensure that the defined point (e.g., trace peak) of aortic valve closure is correct. The AVC timing also may be automatically confirmed, for example, by comparison to an expected value. The AVC may be adjusted as desired or needed.

Thereafter, a parametric image may be generated at 300 in any known manner and displayed. For example, a peak systolic strain image or end systolic strain image with color coded heart wall contraction information may be displayed, which may also include a percentage value of contraction information.

Additional displays may be provided as part of the workflow 290. For example, at 302, strain traces or bullseye plot(s) (as shown in FIG. 6) may be generated and displayed in any known manner and as described herein.

Thus, the various embodiments provide functional ultrasound imaging wherein 2D tracking is based on multi-plane data acquisition, such as in a 3D imaging mode. Accordingly, left ventricle quantification based on 2D speckle tracking in simultaneously or near-simultaneously acquired multi-plane data is provided. The number of acquired apical planes may be increased, for example, by combining or stitching sequentially acquired multi-plane data that may be acquired by electronically rotating the scan angles of a ultrasound probe without moving the ultrasound probe. Additionally, the apical point for all scan planes can be automatically determined (or estimated) based on the left ventricle long axis orientation defined by the multi-plane scan.

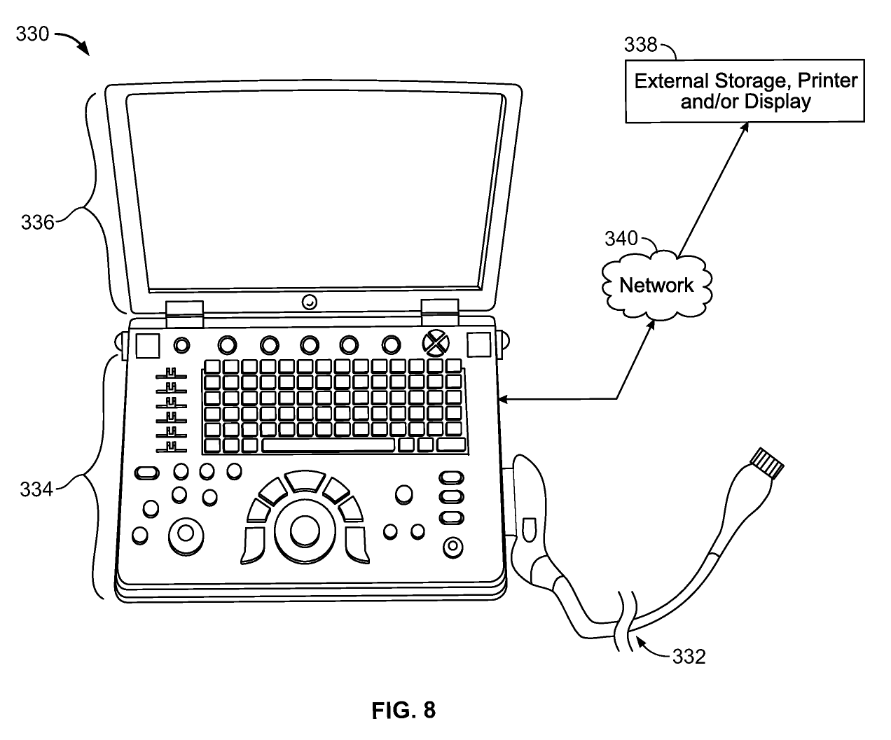

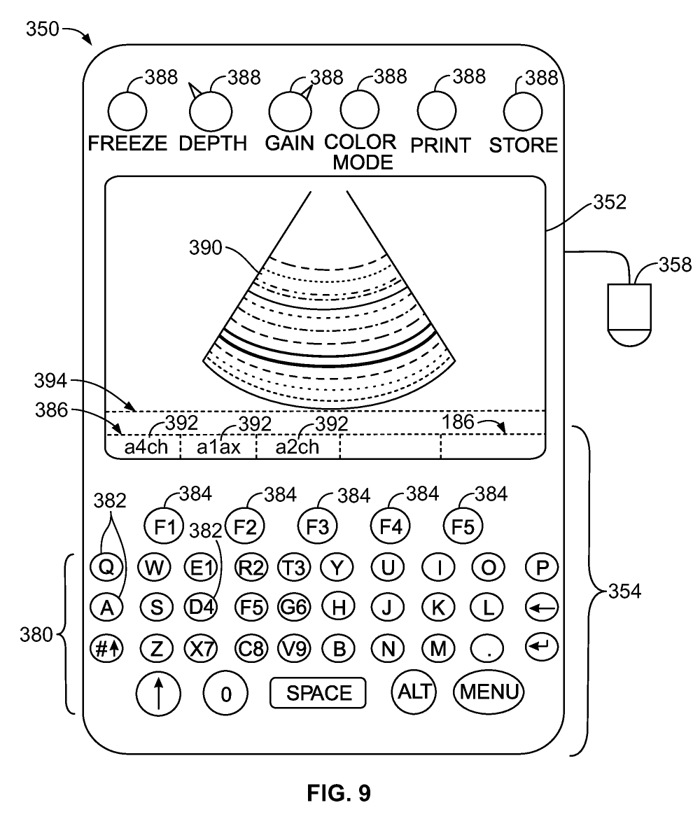

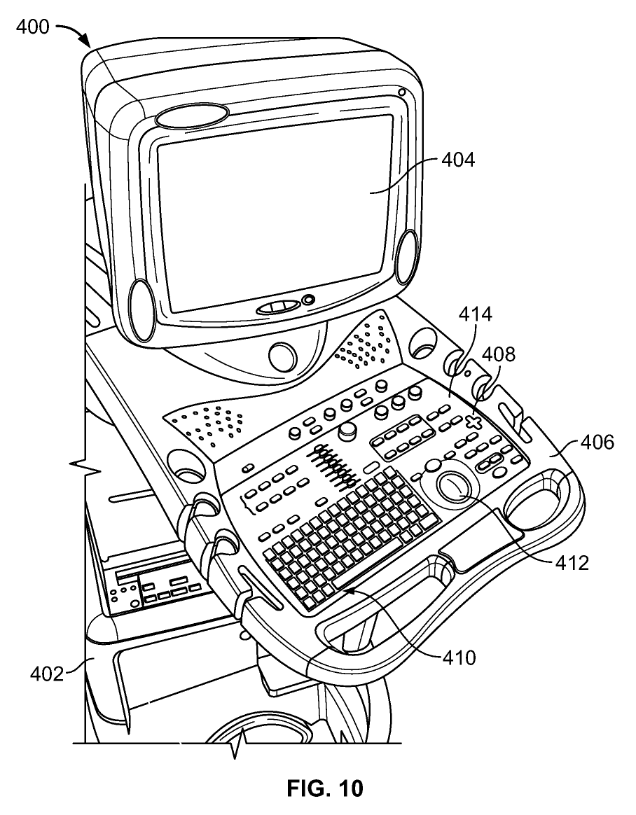

The ultrasound system 100 of FIG. 1 may be embodied in a small-sized system, such as laptop computer or pocket sized system as well as in a larger console-type system. FIGS. 8 and 9 illustrate small-sized systems, while FIG. 10 illustrates a larger system.

FIG. 8 illustrates a 3D-capable miniaturized ultrasound system 330 having a probe 332 (e.g., a three-dimensional (3D) transesophageal echocardiography (TEE) ultrasound probe) that may be configured to acquire 3D ultrasonic data, namely multi-plane ultrasonic data. For example, the probe 332 may have a 2D array of elements 104 as discussed previously with respect to the probe 106 of FIG. 1. A user interface 334 (that may also include an integrated display 336) is provided to receive commands from an operator. As used herein, “miniaturized” means that the ultrasound system 330 is a handheld or hand-carried device or is configured to be carried in a person's hand, pocket, briefcase-sized case, or backpack. For example, the ultrasound system 330 may be a hand-carried device having a size of a typical laptop computer. The ultrasound system 330 is easily portable by the operator. The integrated display 336 (e.g., an internal display) is configured to display, for example, one or more medical images.

The ultrasonic data may be sent to an external device 338 via a wired or wireless network 340 (or direct connection, for example, via a serial or parallel cable or USB port). In some embodiments, the external device 338 may be a computer or a workstation having a display. Alternatively, the external device 338 may be a separate external display or a printer capable of receiving image data from the hand carried ultrasound system 330 and of displaying or printing images that may have greater resolution than the integrated display 336.

FIG. 9 illustrates a hand carried or pocket-sized ultrasound imaging system 350 wherein the display 352 and user interface 354 form a single unit. By way of example, the pocket-sized ultrasound imaging system 350 may be a pocket-sized or hand-sized ultrasound system approximately 2 inches wide, approximately 4 inches in length, and approximately 0.5 inches in depth and weighs less than 3 ounces. The pocket-sized ultrasound imaging system 350 generally includes the display 352, user interface 354, which may or may not include a keyboard-type interface and an input/output (I/O) port for connection to a scanning device, for example, an ultrasound probe 356. The display 352 may be, for example, a 320×320 pixel color LCD display (on which a medical image 190 may be displayed). A typewriter-like keyboard 380 of buttons 382 may optionally be included in the user interface 354.

Multi-function controls 384 may each be assigned functions in accordance with the mode of system operation (e.g., displaying different views). Therefore, each of the multi-function controls 384 may be configured to provide a plurality of different actions. Label display areas 386 associated with the multi-function controls 384 may be included as necessary on the display 352. The system 350 may also have additional keys and/or controls 388 for special purpose functions, which may include, but are not limited to “freeze,” “depth control,” “gain control,” “color-mode,” “print,” and “store.”

One or more of the label display areas 386 may include labels 392 to indicate the view being displayed or allow a user to select a different view of the imaged object to display. For example, the labels 392 may indicate an apical 4-chamber view (a4ch), an apical long axis view (a1ax) or an apical 2-chamber view (a2ch). The selection of different views also may be provided through the associated multi-function control 384. For example, the 4ch view may be selected using the multi-function control F5. The display 352 may also have a textual display area 394 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 350 and the miniaturized ultrasound system 330 of FIG. 8 may provide the same scanning and processing functionality as the system 100 (shown in FIG. 1).

FIG. 10 illustrates a portable ultrasound imaging system 400 provided on a movable base 402. The portable ultrasound imaging system 400 may also be referred to as a cart-based system. A display 404 and user interface 406 are provided and it should be understood that the display 404 may be separate or separable from the user interface 406. The user interface 406 may optionally be a touchscreen, allowing the operator to select options by touching displayed graphics, icons, and the like.

The user interface 406 also includes control buttons 408 that may be used to control the portable ultrasound imaging system 400 as desired or needed, and/or as typically provided. The user interface 406 provides multiple interface options that the user may physically manipulate to interact with ultrasound data and other data that may be displayed, as well as to input information and set and change scanning parameters and viewing angles, etc. For example, a keyboard 410, trackball 412 and/or multi-function controls 414 may be provided.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for functional ultrasound imaging, the method comprising:
obtaining ultrasound image data acquired from a multi-plane imaging scan of an imaged object, the ultrasound image data defining a plurality of image planes;
determining functional image information for the imaged object from two-dimensional tracking information based on the plurality of image planes; and
generating functional ultrasound image data for the imaged object using the functional image information.

2. A method in accordance with claim 1 wherein determining functional image information comprises one of separately or jointly processing each of the plurality of image frames.

3. A method in accordance with claim 1 further comprising performing two-dimensional tracking to determine the functional image information.

4. A method in accordance with claim 1 wherein the plurality of image planes are acquired simultaneously.

5. A method in accordance with claim 1 wherein the plurality of image planes are acquired consecutively within a short period of time.

6. A method in accordance with claim 1 wherein the imaged object is a heart and the ultrasound image data comprises imaged heart data with the functional information comprising myocardium contraction information.

7. A method in accordance with claim 6 further comprising automatically determining an apical point position in each of a plurality of image frames based on an apical point in at least one of the plurality of image frames.

8. A method in accordance with claim 1 wherein the multi-plane imaging scan comprises a tri-plane imaging scan.

9. A method in accordance with claim 8 wherein the tri-plane imaging scan comprises a plurality of apical image planes at different rotated scan angles.

10. A method in accordance with claim 1 wherein the multi-plane imaging scan comprises a plurality of tri-plane imaging scans.

11. A method in accordance with claim 10 wherein the plurality of tri-plane imaging scans are sequentially acquired.

12. A method in accordance with claim 11 further comprising combining imaging data from the plurality of tri-plane imaging scans.

13. A method in accordance with claim 11 wherein the plurality of tri-plane imaging scans comprise a plurality of rotated single plane scans.

14. A method in accordance with claim 11 wherein the plurality of tri-plane imaging scans comprise a plurality of rotated bi-plane scans.

15. A method in accordance with claim 1 further comprising displaying a combined image based on the functional ultrasound image data.

16. A method in accordance with claim 15 wherein the imaged object is a heart and the combined image comprises a graphical representation of a left ventricle of the imaged heart with the graphical representation including the functional image information.

17. A method in accordance with claim 1 wherein the ultrasound image data comprises a three-dimensional (3D) acquisition data and wherein multiplane data is extracted from the 3D acquisition data.

18. A method in accordance with claim 17 wherein an axis for the multiplane data is determined from a scanning axis.

19. A computer readable medium having computer readable code readable by a machine and with instructions executable by the machine to perform a method of functional imaging, the method comprising:
accessing multi-plane ultrasound image data of an imaged object;
performing two-dimensional tracking using the multi-plane ultrasound image data;
determining functional image information based on the two-dimensional tracking; and
generating functional ultrasound image data using the functional image information.

20. A computer readable medium in accordance with claim 19 wherein the imaged object is a heart and the instructions executable by the machine cause the machine to further perform automatic determination of an apical point position in each of a plurality of image frames of the multi-plane ultrasound image data based on an apical point in at least one of the plurality of image frames.

21. An ultrasound imaging system comprising:
an ultrasound probe configured to perform multi-plane ultrasound imaging to acquire a plurality of image frames; and
a processor having a functional imaging module configured to determine functional image information from two-dimensional tracking information for the acquired plurality of image frames and generate functional ultrasound image data.

22. An ultrasound system in accordance with claim 21 wherein the ultrasound probe comprises a three-dimensional probe having an electronically steerable matrix array.

23. An ultrasound system in accordance with claim 21 wherein the ultrasound probe comprises a three-dimensional (3D) transesophageal echocardiography (TEE) ultrasound probe.
